(19) United States
(12) Patent Application Publication
(10) Pub. No.: US 2025/0221439 A1
(43) Pub. Date: Jul. 10, 2025

(54) YEAST EXTRACT WITH FREE GLUTAMATE AND 5'-RIBONUCLEOTIDES

(71) Applicant: DSM IP ASSETS B.V., TE HEERLEN (NL)

(72) Inventors: Elaheh JAMALZADEH, Echt (NL); Tijmen Jasper BLOK, Echt (NL)

(21) Appl. No.: 18/850,760

(22) PCT Filed: Apr. 4, 2023
(86) PCT No.: PCT/EP2023/058753

§ 371 Date: Sep. 25, 2024

(30) Foreign Application Priority Data
Apr. 15, 2022 (EP) 22168625.6

(51) Int. Cl. A23L 31/15; A23L 23/00; A23L 27/22; A23L 27/23; A23L 27/24; C12N 9/16; C12N 9/78

(52) U.S. Cl. CPC A23L 31/15; A23L 23/00; A23L 27/22; A23L 27/235; A23L 27/24; C12N 9/16; C12N 9/78; C12Y 301/04; C12Y 305/04

(57) ABSTRACT

The present invention relates to a yeast extract comprising free glutamate in an amount of at least 7 wt. % on salt free dry matter and comprising an amount of 5′-ribonucleotides of at least 10 wt. % on salt free dry matter.

### FIELD

The present invention relates to a yeast extract comprising free glutamate and 5′-ribonucleotides. Further, the present invention relates to a method to produce a yeast extract. Further, the present invention relates to the use of a yeast extract.

### BACKGROUND

Yeast extracts are known for providing an umami flavour. Free glutamate, or glutamic acid, is a known compound to provide the umami flavour. Further, 5′-ribonucleotides are known for providing flavour enhancement. Free glutamate and 5′-ribonucleotides combined provide synergy in umami flavour strength. Conventional ways to provide this synergy are to combine yeast extracts that are rich in free glutamate with yeast extracts that are rich in 5′-ribonucleotides.

There is a need in the art to provide yeast extracts comprising both free glutamate as well as 5′-ribonucleotides as this would allow a lower dosage of the yeast extract in application. However, in producing such a yeast extract, the challenge is that conventional processes provide either a high amount of free glutamate or a high amount of 5′-ribonucleotides. Therefore, there is a need in the art for a process that provides a yeast extract having both free glutamate as well as 5′-ribonucleotides.

### DETAILED DESCRIPTION

The present invention relates to a yeast extract comprising free glutamate in an amount of at least 7 wt. % on (salt free or NaCl free) dry matter and/or comprising an amount of 5′-ribonucleotides of at least 10 wt. % on (salt free) dry matter (of the yeast extract).

The present inventors found that the yeast extract of the invention provides an umami flavour with a higher strength then blends of yeast extracts rich in free glutamate with yeast extracts rich in 5′-ribonucleotides. Hence, the present yeast extract can surprisingly be dosed in lower amounts to achieve a desired umami flavour as compared to blends of yeast extracts rich in free glutamate with yeast extracts rich in 5′-ribonucleotides. This is advantageous in view of costs in use and that application recipes needs less yeast extract, leaving room for other ingredients.

In a preferred embodiment, the present amount of free glutamate is from 7 to 15 wt. % on salt free dry matter and/or the present amount of 5′-ribonucleotides is from 10 to 20 wt. % on salt free dry matter.

In a preferred embodiment, the amount of free glutamate is at least 8, 9, 10, 11, 12, 13, 14 or at least 15 wt. % on (salt free) dry matter (of the yeast extract).

In a preferred embodiment, the amount of 5′-ribonucleotides is at least 11, 12, 13, 14 or at least 15 wt. % on (salt free) dry matter (of the yeast extract).

Preferably, the present yeast extract comprises at least 8 or 9 wt. % free glutamate on (salt free) dry matter (of the yeast extract) and at least 12 or 13 wt. % 5′-ribonucleotides on (salt free) dry matter (of the yeast extract).

In a preferred embodiment, the present amount of free glutamate is from 9 to 14 wt. % on (salt free) dry matter (of the yeast extract).

Preferably, the present amount of free glutamate is from 7.5 to 14, 8 to 13, 8.5 to 12 or 9 to 11 wt. % on (salt free) dry matter (of the yeast extract).

In a preferred embodiment, the amount of 5′-ribonucleotides is from 10 to 20 wt. % on (salt free) dry matter (of the yeast extract).

Preferably, the present amount of 5′-ribonucleotides is from 11 to 19, 12 to 18, 13 to 17 or 14 to 16 wt. % on (salt free) dry matter (of the yeast extract).

Preferably the present amount of free glutamate is from 7.5 to 14 or 8 to 13 wt. % on (salt free) dry matter (of the yeast extract) and the amount of 5′-ribonucleotides is from 12 to 18 or 14 to 16 wt. % on (salt free) dry matter (of the yeast extract).

The term yeast extract is defined as the water-soluble components of the yeast cell, the composition of which is primarily amino-acids, peptides, carbohydrates and salts. The yeast may be any suitable yeast and is preferable selected from the genera Saccharomyces, Brettanomyces, Kluyveromyces, Candida or Torula, preferably the genus Saccharomyces, more preferably Saccharomyces cerevisiae as is used as a baker's yeast.

The term “5′-ribonucleotides” is herein intended to refer to either the free 5′-ribonucleotides or salts thereof.

In a preferred embodiment, the present 5′-ribonucleotides are 5′-inosine mono phosphate (5′-IMP) and 5′-guanine mono phosphate (5′-GMP). Preferably the present 5′-ribonucleotides consist essentially of 5′-inosine mono phosphate (5′-IMP) and 5′-guanine mono phosphate (5′-GMP). Preferably the present 5′-ribonucleotides are for at least 90, 91, 92, 93, 94, 95, 96, 97, 98 or at least 99 wt. % 5′-inosine mono phosphate (5′-IMP) and 5′-guanine mono phosphate (5′-GMP), of the 5′-ribonucleotides.

In a preferred embodiment, the present yeast extract further comprises salt (NaCl) in an amount from 1 to 20 wt. % on dry matter (of the yeast extract). Preferably, the present yeast extract comprises salt (NaCl) in an amount from 2 to 19, 3 to 18, 4 to 17, 5 to 15 or 6 to 14 wt. % on dry matter (of the yeast extract).

In a preferred embodiment, the present yeast extract has a dry matter of at least 90 wt. %, preferably at least 91, 92, 93, 94, 95, 96, 97, 98 or 99 wt. % (of the yeast extract). Hence, preferably the yeast extract is in a powder form.

Preferably the present yeast extract is packed in bags of 1 to 50 kg, preferably 2 to 30 kg, preferably 5 to 25 kg or preferably 10 to 15 kg.

According to another aspect, the present invention relates to a method to produce the yeast extract as defined herein, comprising the steps of:
(i) subjecting yeast cells to a temperature of at least 80° C. for at least 1 minute to provide yeast cells having inactive yeast native enzymes;
(ii) incubating the yeast cells having inactive native enzymes at a pH from 5 to 10 without the presence of (exogenous) enzymes (or without adding enzymes), for a time period of 1 to 10 hours to provide incubated yeast cells;
(iii) subjecting the incubated yeast cells to 5′-phosphodiesterase to hydrolyze RNA into 5′-ribonucleotides; and
(iv) separating the soluble fraction by solid liquid separation to provide the yeast extract.

Surprisingly, the present inventors found that the present method efficiently provides a yeast that is high in free glutamate and high in 5′-ribonucleotides.

Preferably the pH at step (ii) is from 5.1 to 9, from 5.3 to 8.5, from 5.5 to 8, from 6 to 9 or from 6.5 to 7.5. Preferably the time period is from 2 to 8 hours such as 3 to 6 hours.

Preferably, present step (ii) is carried out at a temperature within the range of 40 to 80° C., such as 45 to 75 or 50 to 70° C.

In an embodiment, present step (iii) is carried out at a temperature within the range of 40 to 80° C. for a time period of 10 to 30 hours, and/or a pH of 5 to 8.

In an embodiment present step (iii) further comprises subjecting the incubated yeast cells to a deaminase, preferably for conversion of 5′-AMP to 5′-IMP.

In an embodiment, the present method further comprises a step (v) of drying the yeast extract. Preferably the drying step is a spray drying step.

According to another aspect, the present invention relates to the use of the present yeast extract for providing an umami taste, preferably for providing an umami taste in food or feed applications. Preferably wherein no other yeast extract is used or preferably wherein the yeast extract is the sole yeast extract.

The invention is further illustrated in the following examples.

### Example 1

### Prepare a Yeast Extract Containing High Levels of Free Glutamate and 5′-Ribonucleotides

5 portions of (2-liter) cream yeast from Saccharomyces cerevisiae were heat treated in for 10 minutes at 95° C. to inactivate all native yeast enzymes. All portions of 2 l of cream yeast (vessel 1, 2, 3, 4, and 5) from Saccharomyces cerevisiae were rapidly cooled down to 61° C. In vessel 1 (control) 0.1 ml Collupoline (commercially available serine protease from DSM N.V. The Netherlands) was added and the mixtures was incubated for 4 hours at pH 5.3 and 61° C.

In 4 other vessels (2,3,4,5), the pH of the heat shocked cream yeast was adjusted to 5.3, 7, 8, 9 using KOH (4M) and H₂SO₄ (2M) and incubated at 61° C. for 4 hours without adding protease. After 4 hours, the condition was adjusted in all vessels to temperature of 61° C. and a pH of 5.3 and treated for 20 hours with 5′-phosphodiesterase (4.4 ml) and Deaminase (0.17 gr) to hydrolyze the RNA into 5′-ribonucleotides. The yeast broth in vessel 1 was warmed up to 60-70° C. for 30 minutes to inactivate the protease. No heat treatment was applied for other vessels. The extract (soluble fraction) was separated from the insoluble cell walls by means of centrifugation and the soluble fraction was analyzed for free glutamate and 5′-ribonucleotides. During the 4 hours incubation of cream yeast, samples are taken and centrifuged, and the RNA is measured in the supernatant. The level of RNA released in time during incubation at different pH is shown in Table 1.

TABLE 1
yield after 4 h incubation and RNA in solution.

| | | Solubilization yield | | | |
|---|---|---|---|---|---|
| | | after 4 hours | RNA in Solution | | |
| | | incubation before | % on dm | | |
| Vessel | pH | RNA hydrolysis | 1 h | 3 h | 4 h |
| 1 | 5.3 | NA | NA | NA | NA |
| | (+protease | | | | |
| | enzyme) | | | | |
| 2 | 5.3 | 34% | NA | NA | 9 |
| 3 | 7 | 38% | 13 | 13 | 14 |
| 4 | 8 | 41% | 15 | 17 | 17 |
| 5 | 9 | 49% | 17 | 18 | 18 |

NA: not analyzed

It is observed that the higher the pH the more RNA is released from the cells. The highest RNA was measured after 4 hours incubation at pH 9.

Data on RNA, solubilization yield, free glutamate, and 5′-ribonucleotides after RNA hydrolysis is presented in table 2.

TABLE 2
Hydrolysis yield and product composition as a function of pH after RNA hydrolysis

| | | | Solubilization | Free | 5′ IMP + | RNA hydrolysis |
|---|---|---|---|---|---|---|
| Vessel | pH | Condition | yield | glutamate | 5′GMP | yield |
| 1 (Ref) | 5.3 | +protease | 54% | 6.7% on dm | 12.7% on dm | 81.9% |
| 2 | 5.3 | No protease | 39% | 9.6% on dm | 13.0% on dm | 59.3% |
| 3 | 7 | No protease | 42% | 8.8% on dm | 13.4% on dm | 66.0% |
| 4 | 8 | No protease | 45% | 8.1% on dm | 13.2% on dm | 69.7% |
| 5 | 9 | No protease | 49% | 5.9% on dm | 12.0% on dm | 70.7% |

The reference experiment led to higher biomass solubilization yield but the level of glutamate and nucleotide was lower. By removing the enzyme at the same pH (5.3), the biomass yield was lower and led to the highest glutamate level. Incubation at pH 8 was the optimum condition in this process where the highest RNA hydrolysis yield was obtained (more RNA was released) and both nucleotides and glutamate were high. At pH 9 (table 1) more RNA is measured in the supernatant, but due to pH adjustment more titrants were used which leads to dilution of amino acids and nucleotides.

### Example 2

### Replacing a High Nucleotide Yeast Extract in Vegetable Bouillon

The yeast extract prepared in example 1 (number 4 in Table 2 with 8.1 wt. % on dm free glutamate and 13.2 wt. % 5′ IMP and 5′ GMP) and a high nucleotide yeast extract containing 14% 5′ IMP and 5′ GMP on salt free dry matter and 3.5% glutamate on dry matter were tested in a vegetable bouillon (Maxarome® Select, from DSM, The Netherlands). This vegetable bouillon was prepared by dry blending all ingredients from Table 3 in tap water of 95° C. and stirred until homogeneity. Products were cooled down until 60° C. before sensorial evaluation. The samples were tested in a blind test by an expert panel for Savoury applications (n=4), which was followed by a discussion. The new prototype was dosed at about 50% of the high nucleotide yeast extract (Maxarome® Select), however the products were found to be almost equal on umami intensity.

TABLE 3
Compositions of bouillon formulations
all numbers in weight (gr)

| | Maxarome ® | YE |
|---|---|---|
| | Select | example 1 |
| Salt | 7.60 | 8.00 |
| Maltodextrin | 3.60 | 4.10 |
| Vegetable fat | 2.70 | 2.70 |
| Maxarome ® Select | 2.00 | |
| Native potato starch | 1.80 | 1.80 |
| Turmeric powder | 0.126 | 0.126 |
| Onion powder | 0.090 | 0.090 |
| Cellery leaf powder | 0.018 | 0.018 |
| White pepper | 0.018 | 0.018 |
| YE example 1 | | 1.10 |
| Water | 1000 | 1000 |

### Example 3

Replacing a High Nucleotide Yeast Extract Combined with High Glutamate in Vegetable Bouillon

The yeast extract prepared in example 1 (number 4 in Table 2 with 8.1 wt. % on dm free glutamate and 13.2 wt. % 5′ IMP and 5′ GMP) and a high nucleotide yeast extract containing 14% I&G on dry matter and 3.5% glutamate on salt free dry matter (Maxarome® Select, from DSM, The Netherlands) combined with a high glutamate yeast extract containing 9% glutamate on salt free dry matter (Gistex® HUM LS, DSM, The Netherlands) were tested in a simple vegetable bouillon. This vegetable bouillon was prepared by dry blending all ingredients from Table 4 in tap water of 95° C. and stirred until homogeneity. Products were cooled down until 60° C. before sensorial evaluation. The samples were tested in a blind test by an expert panel for savoury applications (n=4), which was followed by a discussion.

The products were found to be almost equal umami intensity, the new Prototype was found to be cleaner in taste than the combination of Maxarome® Select and Gistex® HUM LS.

TABLE 4
Compositions of bouillon formulations
all numbers in weight (gr)

| | Yeast | YE |
|---|---|---|
| | extracts | example 1 |
| Salt | 7.80 | 8.00 |
| Vegetable fat | 2.70 | 2.70 |
| Maltodextrin | 2.45 | 3.75 |
| Gistex ® HUM LS | 2.00 | |
| Native potato starch | 1.80 | 1.80 |
| YE example 1 | | 1.50 |
| Maxarome ® Select | 1.00 | |
| Turmeric powder | 0.13 | 0.13 |
| Onion powder | 0.09 | 0.09 |
| Cellery leaf powder | 0.02 | 0.02 |
| White pepper | 0.02 | 0.02 |
| Salt | 7.80 | 8.00 |
| Water | 1000 | 1000 |

1. A yeast extract comprising free glutamate in an amount of at least 7 wt. % on salt free dry matter and comprising an amount of 5′-ribonucleotides of at least 10 wt. % on salt free dry matter.

2. The yeast extract according to claim 1, wherein the amount of free glutamate is from 7 to 15 wt. % on salt free dry matter and wherein the amount of 5′-ribonucleotides is from 10 to 20 wt. % on salt free dry matter.

3. The yeast extract according to claim 1, wherein the amount of free glutamate is from 9 to 14 wt. % on salt free dry matter.

4. The yeast extract according to claim 1, wherein the 5′-ribonucleotides are 5′-inosine mono phosphate (5′-IMP) and 5′-guanine mono phosphate (5′-GMP).

5. The yeast extract according to claim 1, further comprising salt in an amount from 1 to 20 wt. % on dry matter.

6. The yeast extract according to claim 1, having a dry matter of at least 90 wt. %.

7. A method to produce the yeast extract as defined in claim 1, comprising:
(i) subjecting yeast cells to a temperature of at least 80° C. for at least 1 minute to provide yeast cells having inactive yeast native enzymes;
(ii) incubating the yeast cells having inactive native enzymes at a pH from 5 to 10 without the presence of enzymes, for a time period of 1 to 10 hours to provide incubated yeast cells;
(iii) subjecting the incubated yeast cells to 5′-phosphodiesterase to hydrolyze RNA into 5′-ribonucleotides; and
(iv) separating the soluble fraction by solid liquid separation to provide the yeast extract.

8. The method according to claim 7, wherein (ii) is carried out at a temperature within a range of 40 to 80° C.

9. The method according to claim 7, wherein (iii) is carried out at a temperature within a range of 40 to 80° C. for a time period of 10 to 30 hours, and a pH of 5 to 8.

10. The method according to claim 1, wherein (iii) further comprises subjecting the incubated yeast cells to a deaminase.

11. The method according to claim 1, further comprising (v) drying the yeast extract.

12. A product comprising a yeast extract as defined in claim 1, for providing an umami taste.

13. The product according to claim 12, for providing an umami taste in one or more food or feed applications.
